(19) United States
(12) Patent Application Publication
(10) Pub. No.: US 2019/0352615 A1
(43) Pub. Date: Nov. 21, 2019

(54) METHOD FOR PURIFYING VIRUS

(71) Applicant: Blue Sky Vaccines GmbH, Vienna (AT)

(72) Inventors: Markus WOLSCHEK, Vienna (AT); Manfred REITER, Vienna (AT)

(21) Appl. No.: 16/472,182

(22) PCT Filed: Dec. 21, 2017
(86) PCT No.: PCT/EP2017/084140

§ 371 Date: Jun. 20, 2019

(30) Foreign Application Priority Data
Dec. 22, 2016 (EP) 16206118.8

(51) Int. Cl. C12N 7/00

(52) U.S. Cl. CPC C12N 7/00; C12N 2760/16351; C12N 2760/16251; C12N 2760/16151

(57) ABSTRACT

The present invention provides a method for purifying virus particles from host cells infected with virus, wherein the virus particles are treated with benzonase and polyethylene glycol and the use of said purified viruses for therapeutic compositions.

The present invention provides a method for purifying virus particles from host cells infected with virus, wherein the virus particles released therefrom are treated with benzonase and polyethylene glycol and the use of said purified viruses for therapeutic compositions.

### BACKGROUND

Commercial production of viral vaccines typically requires large quantities of virus. The development of cell culture-based technologies as an alternative to the traditional egg-based production systems for the manufacture of viral vaccines appears as the most rapid and most promising solution to overcome drawbacks and constraints associated with egg-based production systems.

After production, whether produced on eggs or in cell culture, the produced virus must be recovered from the cell culture, and, when appropriate, be purified.

Efficient vaccine production thus requires the growth of large scale quantities of virus produced in high yields from a host system. Although the cultivation conditions under which a virus is grown is of great significance with respect to achieving an acceptable high yield of the virus, the multistep purification of virus is often accompanied by loss of significant amounts of the virus material and is thus a crucial step for enabling a successful virus vaccine production.

Specifically, influenza epidemic or even pandemic has a high economic impact. Although precise data on influenza associated deaths are not available for all countries, in the United States influenza associated deaths range between 30 and 150 per 100 000 population aged over 65. During influenza epidemics attack rates of 1%-5% are most common, but the attack rate may reach 40%-50%. Vaccination was recognized as the best approach to limit the epidemics and its harmful effects.

Influenza viruses belong to the family Orthomyxoviridae and are separated into types A, B and C according to antigenic differences. Influenza A and B viruses are important respiratory pathogens, although influenza A viruses are the main cause of epidemics with high mortality rate.

Influenza viruses have a segmented, single-stranded RNA genome of negative polarity. The genome of influenza A viruses consists of eight RNA molecules encoding eleven (some influenza A strains ten) proteins. The viral envelope is composed of a lipid bilayer on a layer of matrix protein (M1). Embedded in the lipid bilayer are glycoproteins hemagglutinin (HA) and neuraminidase (NA), which are responsible for virus attachment and penetration into the cell, and release of progeny virus from the infected cells, respectively. Both proteins also determine the subtypes of the influenza A viruses. High mutation rates and frequent genetic reassortment due to the segmented genome, contribute to great immunological variability, particularly of the HA and NA antigens of influenza A viruses. Type B viruses do not exhibit the same degree of antigenic variation and are primarily childhood pathogens, which can occasionally cause epidemics of the same severity as type A.

New influenza epidemics and pandemic are likely to occur in the future and current egg-based vaccine production technology seems to be unable to respond to a pandemic crisis. Thus, a system that can rapidly produce new influenza vaccine is needed. One such approach is a cell culture-based process which can be easily scaled up. But the existing purification of influenza virus vaccine cannot follow this demand. Recently, scalable influenza virus purification process comprising of depth filtration, inactivation, ultra filtration and gel filtration was also described (Nayak D P et al, J. Chromatogr. B Analyt Technol Biomed Life Sci, 823(2): 75-81, 2005). Since virus was inactivated, applicability of this process for purification of infective influenza virus remains unknown.

The use of 5% polyethylene glycol for influenza virus precipitation has been described in U.S. Pat. No. 3,989,818 wherein it is emphasized that higher PEG concentration is a disadvantage making the separation phase difficult due to an increased viscosity.

As seen from above, various different methods for purifying virus have already been developed, however, there is still a high and unmet demand in efficient and economic virus purification which results in increased amounts of virus which is free from contaminating host cell protein and host cell DNA. Therefore, a need remains for providing methods to produce and to purify viruses with an adequate purity level for meeting the regulatory requirements and a good yield at large scale.

### SUMMARY OF THE INVENTION

The problem is solved by the embodiments of the invention.

According to the embodiment of the invention there is provided a method for removal of host cell contamination, specifically host cell proteins and nucleic acids being the most relevant contaminants. DNA contamination as well as other impurities is an enormous challenge that must be solved before viral vaccines from any continuous cell line can be produced at large scale, which is required for global vaccination against highly destructive infectious diseases. Thus the invention contributes to solutions of a significant challenge in the field of live vaccine and/or virus vector production from a continuous cell line.

The method of the invention is amenable to a wide range of viruses.

The inventive method contemplates the purification of enveloped and non-enveloped viruses that leave infected cells by budding from the cell membrane and/or by lysing cells (by destruction of the cells), in particular RNA viruses such as, but not limited to orthomyxoviruses, e.g. influenza virus, paramyxoviruses, e.g. measles virus, togaviruses, e.g. rubella virus, rhabdoviruses, e.g. vesicular stomatitis viruses or rabies viruses, arenaviruses such as lymphocytic choriomeningitis viruses, poxviruses, retroviruses, reoviruses, e.g. rotaviruses. coronaviruses, e.g. SARS corona virus, flaviviridae, e.g. japanese encephalitis virus, yellow fever virus, or Dengue virus, picornaviruses, e.g. polioviruses.

The inventive method also contemplates the purification of enveloped and non-enveloped viruses that leave infected cells by budding from the cell membrane and/or by lysing cells (by destruction of the cells), in particular DNA viruses such as, but not limited to hepadnaviruses, e.g. Hepatitis B virus and parvoviruses.

According to an embodiment of the invention, the method is specifically suitable for purifying lytic virus and viruses budding from the cell membrane.

According to a preferred embodiment, the method is used for influenza virus purification.

The present invention provides a method for purifying virus particles from host cells infected with virus, comprising the sequential steps:

a) providing host cells infected with virus, wherein said cells are releasing virus into cell culture medium,

b) centrifuging the cell culture medium containing said cells,

c) separating supernatant comprising virus particles from precipitate containing cell debris and cells,

d) incubating said supernatant with a nuclease,

e) precipitating the virus particles with polyethylene glycol (PEG),

f) centrifuging the virus particles,

g) re-suspending precipitated virus particles.

In a specific embodiment of the invention, the host cells are virus permeable.

According to an embodiment of the invention, a method is provided for purifying virus particles from host cells infected with virus, comprising the steps in the indicated order:

a) cultivating said host cells under conditions wherein virus particles are released into cell culture,

b) separating supernatant comprising virus particles from cell debris and cells by centrifugation,

c) incubating said supernatant with a nuclease,

d) precipitating the virus particles with polyethylene glycol (PEG),

e) centrifuging the virus particles,

f) re-suspending precipitated virus particles.

According to a further embodiment of the invention, the nuclease is an endonuclease, specifically it is Benzonase®.

In one embodiment, the virus purified and produced by the method of the invention is an RNA virus, specifically belonging to the family of orthomyxoviruses, in particular, influenza virus, more particularly influenza A, B or C virus, more specifically human or avian influenza virus.

In a specific embodiment, the host cells are applied to the purification method as described herein without previous lysis treatment.

According to a further embodiment, the centrifugation is performed with a speed ranging from 1,500 g to 15,000 g, specifically at a range of 2,000 to 10,000 g. According to an embodiment, centrifugation is performed at a speed of at least 2,000 g, specifically at least 3,000 g, specifically at least 4,000 g, specifically centrifugation is at about 4,100 g.

In a specific embodiment of the invention, at least 7.5%, specifically more than 7.5%, specifically at least 8% PEG (w/v) is used in the precipitation step.

In an alternative embodiment, at least 5% PEG (w/v) is used in the precipitation step.

In a specific embodiment of the invention up to 30% PEG (w/v), specifically up to 20% PEG (w/v) is used for virus precipitation, specifically between 7.5% and 15% PEG (w/v), specifically between 7,5% and 10% PEG (w/v).

According to a further aspect of the invention, virus precipitation is performed with 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 26%, 26.5%, 27%, 27.5%, 28%, 28.5%, 29%, 29.5%, 30% w/v PEG.

In an embodiment of the invention, PEG has an average molecular weight from about 5,000 (PEG5000) grams per mole to about 100,000 (PEG100000) grams per mole, specifically from about 5,000 (PEG5000) g/M to about 5,000 (PEG50000) g/M, specifically from about 5,000 (PEG5000) g/M to about 10,000 (PEG10000) g/M, specifically the PEG has an average molecular weight of about 6,000 (PEG 6000) g/M.

In an embodiment of the invention, virus precipitation is free of sodium chloride.

In an alternative embodiment, the virus is precipitated with PEG in the presence of sodium chloride.

In a further embodiment, the virus infected host cells and the virus are not freeze thawn during the purification method as described herein.

With the method of the invention host cell protein removal of at least 50%, specifically at least 60%, specifically at least 70%, specifically at least 80%, specifically at least 85%, specifically at least 90%, specifically at least 95%, more specifically at least 99% can be performed.

With the method of the invention host cell DNA removal of at least 50%, specifically at least 60%, specifically at least 70%, specifically at least 80%, specifically at least 85%, specifically at least 90%, specifically at least 95%, more specifically at least 99% can be performed.

According to a specific embodiment, the resuspended virus is sterile filtered.

According to a further embodiment, the host cells are mammalian cells, specifically Vero cells, HEK-293 and avian cells.

According to a further embodiment, a vaccine is provided comprising virus particles as obtained by the method of the invention, optionally together with further excipients or adjuvants.

In a further embodiment, treatment or prophylactic treatment, specifically vaccination, is provided using the virus particles obtained by the inventive method.

### FIGURES

FIG. 1: Schematic illustration of purification protocol

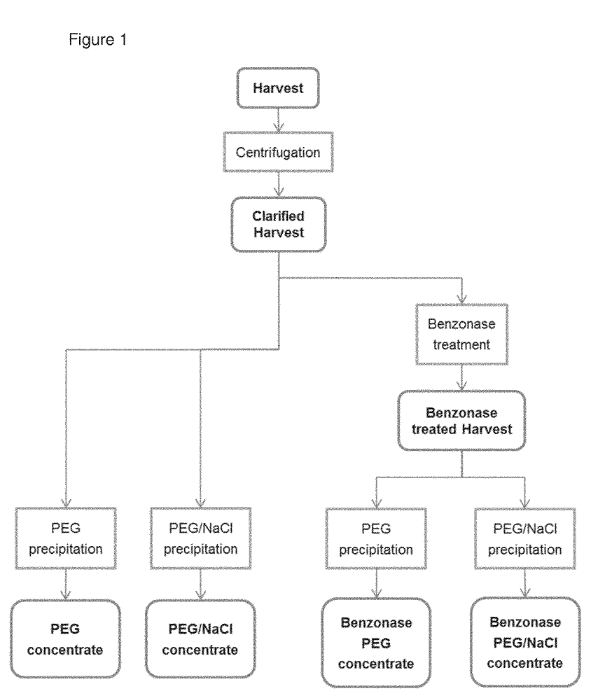

FIG. 2: Comparison of recovery of Influenza Type A deINS 106-P2A-GFP using PEG, PEG/NaCl, Benzonase+PEG, Benzonase+NaCl

FIG. 3: Comparison of recovery of Influenza Type B deINS 106-P2A-GFP using PEG, Benzonase+PEG

FIG. 4: Recovery of Influenza A deINS106-P2A-E6E7 using Benzonase+PEG

### DETAILED DESCRIPTION OF THE INVENTION

Elements of the invention are listed with specific embodiments, however, it should be understood that they may be combined in any manner and in any number to create additional embodiments. The examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. “Consisting” is considered as a closest definition without further elements of the consisting definition feature. Thus “comprising” is broader and contains the “consisting” definition.

As used in this specification and the appended claims, the singular forms “a”, “an”, and “the” include plural referents, unless the content clearly dictates otherwise.

The term “about” as used herein refers to the same value or a value differing by +/−10%, specifically by +/−5% of the given value.

According to a specific embodiment, the host cells are harvested at a time point where the host cells are still largely intact, i.e. when only little or no lysis of the host cell has occurred, and are applied to centrifugation for gaining the virus containing supernatant. Lysis of host cells may naturally occur during propagation, e.g. due to lysis during viral budding or because of senescence and/or apoptosis. Thereby, complex formation between contaminants and viral envelope can also be prevented.

The term “virus permeable host cells” as used therein refers to host cells releasing viruses from the cytoplasm, at least to a certain extent, into the cell culture medium without lysis of the host cells.

As used herein, the term “permeable” refers to permeability of the host cell's cytoplasmic membrane with regard to the release of virus particles. Thereby the viral progeny can cross the cytoplasmic membrane and cell envelope to reach the surrounding medium without the use of a helper virus, freeze thawing or sonication.

The term “incubate” or “contact” as used herein refers to contacting virus containing solutions or media contaminated with host cell components with an excipient, for example, but not limited to, nucleases, DNAses, proteases, enzymes, either naturally occurring or synthetic ones, for a period of time sufficient to achieve a desired effect, e.g. lysis or degradation of host cell compounds like nucleic acids, peptides, polypeptides etc.

Centrifugation conditions as used herein are standard conditions. It is within the skilled person's abilities to determine the appropriate conditions, such as for example the centrifugal force, the operation time, the flow rate speed, or the banding time, if appropriate. The determination of said conditions shall take into account the type of virus-containing fluid to be purified, the type of virus, the type of contaminating impurities, the mode of centrifugation, whether in batch or in continuous, the type of separation, whether zonal or isopycnic. In particular, information provided in the manufacturer's instructions as how to use rotors and centrifuges may guide the skilled person in the selection of appropriate centrifugation conditions. As described earlier, the skilled person may use any standard techniques of protein detection, such as a Western-blot analysis using an antibody specific for a viral antigen, or in the particular case for the influenza virus, the SRI D assay in order to detect the presence of the virus and/or monitor whether acceptable virus yield and/or virus purity after the centrifugation on a pre-formed linear density gradient of the invention are obtained.

Centrifugation is performed to remove cellular cells and cellular contaminants like cell debris and large membrane vesicles, for example by applying low speed centrifugation.

Centrifugation may be performed in a continuous mode, in a semi-continuous mode or in successive batches. In one embodiment, the centrifugation during the method of purifying a virus according to the invention is performed in a continuous mode. Centrifugation in a continuous mode is advantageously used at large scale, when large volumes of virus-containing fluids need to be processed and purified.

The appropriate centrifugal force may be chosen by the skilled person and may depend on the type of rotor and centrifuge used. As non-limiting examples, a centrifugal force ranging from at least 1,000 g to 15,000 g, suitably ranging from 1,500 g to 10,000 g suitably ranging from 1,500 g to 5,000 g, suitably ranging from 2,000 g to 4,500 g, suitably ranging from 4,000 g to 4,500 g specifically being about 4,100 g is applied to the rotor in the centrifuge. The centrifugation time can be easily determined by a skilled person, e.g. by measuring the amount of virus collected by centrifugation. The centrifugation time can range from at least 5 min to 60 min, suitably ranging from 10 min to 40 min, suitable ranging from 15 min to 30 min, specifically being about 15 min.

Once all the virus-containing supernatant or the virus particles have been processed, and an adequate operation time is allowed, the centrifugation is stopped and the virus is collected.

The virus to be purified according to the method of the invention may be produced on cell culture. The method according to the present invention is applicable to any type of suitable host cells, whether adherent cells grown on micro-carriers or suspension cells.

Host cells may be grown in various ways, such as for example, using batch, fed-batch, or continuous systems, such as perfusion systems. Perfusion is particularly advantageous when high cell density is desired. High cell density may be particularly advantageous in order to maximize the amount of virus which can be produced from a given cell type.

The cells used to produce a virus-containing fluid to be purified according to the method of the invention can in principle be any desired type of animal cells which can be cultured in cell culture and which can support virus replication. They can be either primary cells or continuous cell lines. Genetically stable cell lines are preferred. Mammalian cells are particularly suitable, for example, human, hamster, cattle, monkey or dog cells. Alternatively, insect cells are also suitable, such as, for instance, SF9 cells or Hi-5 cells.

Within the scope of the invention, the term “cells” or “host cells” means the cultivation of individual cells, tissues, organs, insect cells, avian cells, mammalian cells, hybridoma cells, primary cells, continuous cell lines, and/or genetically engineered cells, such as recombinant cells expressing a virus. These can be for example BSC-1 cells, LLC-MK cells, CV-1 cells, CHO cells, COS cells, murine cells, human cells, HeLa cells, 293 cells, Vero cells, MDBK cells, MDCK cells, MDOK cells, CRFK cells, RAF cells, TCMK cells, LLC-PK cells, PK15 cells, WI-38 cells, MRC-5 cells, T-FLY cells, BHK cells, SP2/0 cells, NSO, PerC6 (human retina cells), chicken embryo cells or derivatives, embryonated egg cells, embryonated chicken eggs or derivatives thereof. Preferably the cell line is a Vero cell line.

A number of mammalian cell lines are known in the art and include Vero cells (anchorage dependent or suspension grown), PER.C6, HEK cells, human embryonic kidney cells (293 cells), HeLa cells, CHO cells, avian cells (continuous or primary) and MDCK cells which are preferred for the method of the invention.

Suitable monkey cells are, for example, African green monkey cells, such as kidney cells as in the Vero cell line. Suitable canine cells are, for example, kidney cells as in the MDCK cell line.

Suitable mammalian cell lines for growing influenza virus also include PER.C6 cells. These cell lines are all widely available, for instance, from the American Type Cell Culture (ATCC) collection. Alternatively, cell lines for use in the invention may be derived from avian sources, such as chicken, duck, goose, quail or pheasant. Avian cell lines may be derived from a variety of developmental stages including embryonic, chick and adult. In particular, cell lines may be derived from the embryonic cells, such as embryonic fibroblasts, germ cells, or individual organs, including neuronal, brain, retina, kidney, liver, heart, muscle, or extraembryonic tissues and membranes protecting the embryo. Chicken embryo fibroblasts (CEF) and continuous avian cell lines may be used.

According to a particular embodiment, the method of the invention, when the virus to be purified is produced on cell culture, uses Vero cells.

According to a specific embodiment, cultivation of the virus infected host cells is stopped at a time point wherein at least 20%, specifically at least 30%, specifically at least 40%, specifically at least 50%, specifically at least 60%, specifically at least 70%, specifically at least 80%, specifically at least 90%, specifically at least 95% of the host cells are intact and are not destroyed due to virus replication and virus release.

Methods for cell viability testing and quantification methods are well known in the art and can be selected and performed by a skilled person without undue burden. As an example, but not limited thereto, trypan blue dye exclusion cell quantitation and viability assays, fluorometric assays or simultaneous staining procedures can be used (Altman S. A. et al., 1993, Biotechnol.Prog., 9, 671-674).

Quantification of the percentage of intact cells can specifically be performed by a trypan blue exclusion assay. For static cultures and bioreactors the percentage of intact cells (i.e. cells that are not stained by trypan blue) at the time of virus harvest can be determined relative to the number of intact cells in a non-infected culture grown in parallel (i.e. number of intact cells in the infected culture divided by the number of intact cells in a non-infected parallel culture). Alternatively, in bioreactors the percentage of intact cells at the time of virus harvest can be determined as percentage relative to the maximum cell number measured during cell growth phase in the same bioreactor.

Specifically, the host cells are provided to centrifugation for virus particle isolation without any previous lysis treatment, e.g. the host cells are not treated with enzymes, nucleases, proteases and are not freeze thawn, i.e. frozen at temperatures below −20° C. in combination with thawing at temperatures of more than 4° C., or subjected to ultrasonification.

Host cells can be infected with virus with any method known in the art using appropriate moi (multiplicity of infection), specifically in a range of 0.0001 to 0.1.

The term “precipitating” refers to precipitating virus particles from the supernatant. Specifically, precipitation by addition of polyethylene glycol to the virus supernatant is performed. The applicable time and temperature for precipitating virus can be tested and determined by the skilled person. As an example, but not limited thereto, the virus suspension is incubated with PEG for a period of 1 to 48 hours, specifically for a period of 5 to 36 hours, specifically for a period of 10 to 26 hours, specifically about 24 hours.

Polyethylene glycol (PEG) as used in the method of the invention may have a molecular weight in the range of 3,000 to 100,000 g/M, specifically a range of 4,000 to 18,000, 5,000 to 15,000, specifically a molecular weight of about 6000 g/M.

The method of the invention may be employed by a PEG concentration of >5%, specifically in the range of 5 to 20%, specifically in the range of 6 to 10%, specifically at about 8%.

Virus particles purified according to the method as described herein can be suspended in an appropriate solution, specifically a buffer, e.g. but not limited to, virus reconstitution buffer, e.g. a phosphate, Tris.HCl or Hepes based buffer containing e.g. NaCl, sucrose, amino acids, protein hydrolysates, or albumin or any combination of the foregoing.

At the end of the purification, the virus preparation obtained according to the method of the present invention can be directly formulated as virus composition. As an alternative, the virus particles may be subject to sterile filtration, as is common in processes for pharmaceutical grade materials, such as immunogenic compositions or vaccines, and known to the person skilled in the art. Such sterile filtration can for instance suitably be performed by filtering the preparation through a 0.22 μm filter. After sterile preparation, the virus or viral antigens are ready for clinical use, if desired.

The virus purified according to the method of the invention may suitably be formulated to be included in an immunogenic composition, such as a vaccine. Accordingly, a method for the preparation of a vaccine, such as an influenza virus vaccine, comprising at least the step of purifying a virus to be used as an antigen in the vaccine according to the method of the invention and formulating said purified virus into a vaccine is also contemplated in the present invention.

The immunogenic compositions, in particular vaccines, may generally include a whole virus, e.g. a live attenuated whole virus, reasserted virus or an inactivated whole virus.

All viruses amenable for purification according to the inventive method shall be encompassed herein. The viruses may be enveloped viruses, specifically they are RNA viruses.

Virions often nonspecifically associate with cellular components. The accessible surface of infectious viruses for this interaction can be either purely proteinaceous, for example adenoviruses and picornaviruses, or based on a lipid membrane, for example alphavirus, rabies and vaccinia viruses, influenza viruses. The latter are termed enveloped viruses, and for this group of viruses purification is especially difficult because the viral envelope may contain a highly complex and mobile collection of disparate molecules that range from sulfogroups in glycoproteins to aliphatic alcohols in sphingolipids that each or in combination present a range of electrostatic, van der Waals, or hydrophobic interaction surfaces for various binding partners derived from the culture medium itself, producer cells, or other viral particles.

The RNA viruses suitable for the purification according to the invention may be Orthomyxovirus, Flavivirus, Togavirus, Coronavirus, Hepatitis virus, Paramyxovirus, Rhabdovirus, Bunyavirus, Filovirus.

Specifically the viruses are lytic viruses.

The term “lytic virus” shall refer viruses that destroy (lyse) the host cell in course of their replication cycle, i.e. during replication.

According to a specific embodiment, influenza virus is cultured in the host cells and purified according to the method described herein.

The influenza virus can be selected from the group of human influenza virus, avian influenza virus, equine influenza virus, swine influenza virus, feline influenza virus. Influenza virus is more particularly selected in strains A, B and C, preferably from strains A and B. Influenza antigens may be derived from interpandemic (annual or seasonal) influenza strains. Alternatively, influenza antigens may be derived from strains with the potential to cause a pandemic outbreak; i.e., influenza strains with new hemagglutinin compared to hemagglutinin in currently circulating strains, or influenza strains which are pathogenic in avian subjects and have the potential to be transmitted horizontally in the human population or influenza strains which are pathogenic to humans. Depending on the particular season and on the nature of the antigen included in the vaccine, the influenza antigens may be derived from one or more of the following hemagglutinin subtypes: H1, H2, H3, H4, H5, H6, H7, H8, H9, H10, H11, H12, H13, H14, H15 or H16. Preferably, the influenza virus or antigens thereof are from H1, H2, H3, H5, H7 or H9 subtypes. In one embodiment, the influenza viruses are from H2, H5, H6, H7 or H9 subtypes. In an alternative embodiment, the influenza viruses are from H1, H3 or B subtypes.

According to a specific embodiment, the influenza virus is an attenuated influenza virus. Specifically, the influenza virus comprises deletions or modifications within the pathogenicity factors inhibiting innate immune response of host cells. The attenuation can exemplarily be derived from cold-adapted virus strains or due to a deletion or modification within the NS1 gene (ΔNS1 virus) as described in WO99/64571 and WO99/64068 which are incorporated herein in total by reference.

Specifically, the influenza virus vector comprises a truncated NS1 protein that contains up to 122 amino acids, preferably up to 121 amino acids, preferably up to 120 amino acids, preferably up to 119 amino acids, preferably up to 118 amino acids, preferably up to 117 amino acids, preferably up to 116 amino acids, preferably up to 115 amino acids, preferably up to 114 amino acids, preferably up to 113 amino acids, preferably up to 112 amino acids, preferably up to 111 amino acids, preferably up to 110 amino acids, preferably up to 109 amino acids, preferably up to 108 amino acids, preferably up to 107 amino acids, preferably up to 106 amino acids, preferably up to 105 amino acids, preferably up to 104 amino acids, preferably up to 103 amino acids, preferably up to 102 amino acids, preferably up to 101 amino acids, preferably up to 100 amino acids, preferably up to 99 amino acids, preferably up to 98 amino acids, preferably up to 97 amino acids, preferably up to 96 amino acids, preferably up to 95 amino acids, preferably up to 94 amino acids, preferably up to 93 amino acids, preferably up to 92 amino acids, preferably up to 91 amino acids, preferably up to 90 amino acids, preferably up to 89 amino acids, preferably up to 88 amino acids, preferably up to 87 amino acids, preferably up to 86 amino acids, preferably up to 85 amino acids, preferably up to 84 amino acids, preferably up to 83 amino acids, preferably up to 82 amino acids, preferably up to 81 amino acids, preferably up to 80 amino acids, preferably up to 79 amino acids, preferably up to 78 amino acids, preferably up to 77 amino acids, preferably up to 76 amino acids, preferably up to 75 amino acids, preferably up to 74 amino acids, preferably up to 73 amino acids of the N-terminus of the NS1 protein.

It was demonstrated that deletion of the NS1 protein or functional knock-out of the protein leads to a significant attenuation of influenza virus due to lack of replication in interferon competent cells or organisms (replication deficient phenotype). Viruses lacking the NS1 protein are not able to antagonize cytokine production of infected cells, therefore inducing self-adjuvanting and immune modulating effects. The hallmark of immune response after immunization with DeINS1 virus is triggering of Th1 type of immune response associated with predominant IgG2A antibody isotype response (B. Ferko et al. 2002).

“Modification” refers to a substitution or deletion of one or more nucleic acids as compared to a wild-type NS1 sequence. Modification within the NS gene can lead to virus particles that are growth deficient in interferon competent cells. Growth deficient means that these viruses are replication deficient as they undergo abortive replication in the respiratory tract of animals. Alternatively, the viruses can comprise deletion or modification of the PB1-F2 gene. The method according to the invention can be specifically used for producing an influenza virus comprising a deletion of the NS1 protein functionality.

The term “NS1 protein functionality” refers to antagonizing capability of inhibit host immune response in cells, especially the limitation of both interferon (IFN) production and the antiviral effects of IFN-induced proteins, such as dsRNA-dependent protein kinase R (PKR) and 2′5′-oligoadenylate synthetase (OAS)/RNase L. A functional NS1 protein provides the virus with the capability to grow in interferon competent cells and to antagonize cytokine production is said cells.

In a specific embodiment, the method of the invention is used for influenza virus purification, wherein said influenza virus additionally expresses a heterologous polypeptide, which can be, but is not limited to tumor associated antigens such as WT1, MUC1, LMP2, HPV E6 and E7, EGFRvIll, MART1 ; MAGE A3, NY ESO1, gp100 and cytokines such as IFN, IL2, IL15, GM-CSF, IL-15, MIP 1alpha and MIP3 alpha or a derivative or fragment thereof.

To allow sufficient cleavage of the heterologous polypeptide, proliferation sequences are additionally inserted at the C-terminal or N-terminal region of the heterologous polypeptide, for example, but not limited to, ubiquitin or picornavirus 2A protein is additionally inserted.

The method of purification according to the invention is particularly suitable for preparing influenza virus immunogenic compositions, including vaccines. Various forms of influenza virus are currently available. They are generally based either on live virus or inactivated virus. Inactivated vaccines may be based on whole virions, split virions or purified surface antigens (including HA). Influenza antigens can also be presented in the form of virosomes (nucleic acid-free viral-like liposomal particles).

Influenza virus strains for use in vaccines change from season to season. Trivalent vaccines are typical, but higher valence, such as quadrivalence, is also contemplated in the present invention. The invention may use HA from pandemic strains (i.e. strains to which the vaccine recipient and the general human population are immunologically naive), and influenza vaccines for pandemic strains may be monovalent or may be based on a normal trivalent vaccine supplemented by a pandemic strain.

Compositions of the invention may include antigen(s) from one or more influenza virus strains, including influenza A virus and/or influenza B virus. In particular, a trivalent vaccine including antigens from two influenza A virus strains and one influenza B virus strain is contemplated by the present invention. Alternatively, a quadrivalent vaccine including antigens from two influenza A virus strains and two influenza B virus strains is also within the scope of the present invention.

The compositions of the invention are not restricted to monovalent compositions, i.e. including only one strain type, i.e. only seasonal strains or only pandemic strains. The invention also encompasses multivalent compositions comprising a combination of seasonal strains and/or of pandemic strains.

Once an influenza virus has been purified for a particular strain, it may be combined with viruses from other strains and/or with adjuvants known by the art.

Any nuclease or endonuclease may be used for incubating the virus particle supernatant according to the invention. As examples, Cyanase™ or Benzonuclease® are well known industrially applicable nucleases. Cyanase™ is a cloned highly active non-Serratia based non-specific endonuclease that degrades single and double stranded DNA and RNA. Benzonase® or Supernuclease as used herein is a nuclease, specifically an endonuclease from Serratia marcescens. The protein is a dimer of 30 kDa subunits with two essential disulfide bonds. This endonuclease attacks and degrades all forms of DNA and RNA (single stranded, double stranded, linear and circular) and is effective over a wide range of operating conditions. The optimum pH for enzyme activity is found to be 8.0-9.2. It completely digests nucleic acids to 5′-monophosphate terminated oligonucleotides 3 to 5 bases in length.

Benzonase® unit definition is as follows: One unit will digest sonicated salmon sperm DNA to acid-soluble oligonucleotides equivalent to a ΔA₂₆₀ of 1.0 in 30 min at pH 8.0 at 37 C (reaction volume 2.625 ml).

The nuclease as used herein may be added to the virus containing host cell supernatant at a concentration of between 10 and 50 U/ml, specifically 15 and 40 U/ml, specifically at about 20 U/ml.

The virus particles obtained by the methods described herein can be used for the preparation of compositions for treatment of individuals, specifically for prophylactic treatment, e.g. by vaccination.

The term “treatment” relates to any treatment which improves the health status, reduces or inhibits unwanted weight loss and/or prolongs and/or increases the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” can be used interchangeably and relate to any treatment that is intended to prevent a disease from occurring in an individual.

As used herein, “preventing” or “prevention” of a disease, disorder or condition refers to the reduction of the occurrence of the disorder or condition in a treated subject relative to an untreated control subject, or delays the onset or reduces the severity of one or more symptoms of the disorder or condition relative to the untreated control subject. The terms “protect”, “prevent”, “prophylactic”, “preventive”or “protective” relate to the prevention and/or treatment of the occurrence and/or the propagation of a disease.

The present invention is further illustrated by the following examples without being limited thereto.

### EXAMPLES

Combination of nuclease treatment and PEG precipitation results in high virus yield with low DNA and protein (host cell protein) impurities. One-step purification with high yield, high potency with low DNA levels that meet regulatory requirements is provided.

Experimental Data

Experiment 1

Influenza A deINS106-P2A-GFP virus was grown in Vero cells seeded in tissue culture flasks. The virus harvest was clarified by centrifugation for 15 min at 4.100 g and 22° C.

The clarified harvest was optionally treated with Benzonase (20 U/ml for 2 h at RT). Virus precipitation was performed by incubation for 24 h at 4° C. with 8% PEG (w/v) in the presence or absence of 100 mM NaCl followed by 30 min centrifugation at 4.100 g and 4° C. Finally, the precipitated virus was resuspended in virus reconstitution buffer (VRB; phosphate pH 7.5, sucrose, arginine) at 1/10th of the initial harvest volume resulting in a 10-fold concentrate.

Analyses

Infectious titers were determined by TCID50 assay in Vero cells. Total protein quantification was done using a Biorad Bradford assay. For each matrix (OptiPro and VRB) an 8-point standard curve was run in parallel. DNA quantification was done in triplicates using a Picogreen assay. For each matrix (OptiPro and VRB) an 8-point standard curve was run in parallel.

TABLE 1

| | TCID50 | Protein | DNA |
|---|---|---|---|
| Titer/concentration | [log/ml] | [μg/ml] | [ng/ml] |
| Harvest | 8.6 | 130 | 6008 |
| Harvest Clarified | 8.6 | 14 | 2512 |
| Harvest | 8.6 | 20 | 363 |
| clarified/Benzonase | | | |
| PEG conc | 9.4 | 251 | 2094 |
| PEG/NaCl conc | 9.7 | 334 | 6284 |
| Benzonase PEG conc | 9.7 | 247 | 490 |
| Benzonase PEG/NaCl | 9.4 | 310 | 476 |
| conc | | | |

TABLE 2

| | TCID50 | Protein | DNA |
|---|---|---|---|
| | recovery | recovery | recovery |
| Recovery | [%] | [%] | [%] |
| Harvest | 100 | 100.0 | 100.0 |
| Harvest Clarified | 84 | 10.9 | 41.8 |
| Harvest | 99 | 15.5 | 6.0 |
| clarified/Benzonase | | | |
| PEG conc | 62 | 19.2 | 3.5 |
| PEG/NaCl conc | 101 | 25.6 | 10.5 |
| Benzonase PEG conc | 115 | 18.9 | 0.8 |
| Benzonase PEG/NaCl | 62 | 23.7 | 0.8 |
| conc | | | |

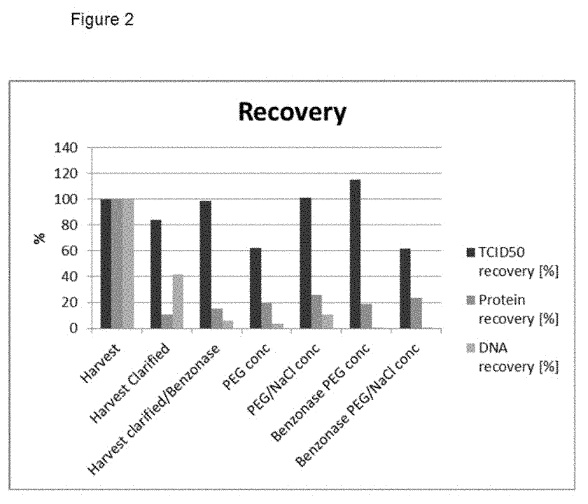

FIG. 2 shows a comparison of recovery using PEG, PEG/NaCl, Benzonase+PEG, Benzonase+NaCl

Experiment 2

Influenza B deINS106-P2A-GFP virus was grown in Vero cells seeded in tissue culture flasks. The virus harvest was clarified by centrifugation for 15 min at 4.100 g and 22° C.

The clarified harvest was optionally treated with Benzonase (20 U/ml for 2 h at RT). Virus precipitation was performed by incubation for 24 h at 4° C. with 8% PEG (w/v) followed by 30 min centrifugation at 4.100 g and 4° C. Each precipitation (+/−Benzonase treatment) was performed in duplicates.

Finally, the precipitated virus was resuspended in virus reconstitution buffer (VRB) at 1/10th of the initial harvest volume resulting in a 10-fold concentrate.

Analyses

Infectious titers were determined by TCID50 assay in Vero cells. Total protein quantification was done using a Biorad Bradford assay. For each matrix (OptiPro and VRB) an 8-point standard curve was run in parallel. DNA quantification was done in triplicates using a Picogreen assay. For each matrix (OptiPro and VRB) an 8-point standard curve was run in parallel.

TABLE 3

| | TCID50 | Protein | DNA |
|---|---|---|---|
| Titer/concentration | [log/ml] | [μg/ml] | [ng/ml] |
| Harvest | 9.0 | 243 | 3524 |
| Harvest Clarified | 8.8 | 56 | 1762 |
| Harvest | 9.0 | 55 | 334 |
| clarified/Benzonase | | | |
| PEG conc 1 | 9.7 | 451 | 5881 |
| PEG/Benzonase conc 1 | 9.6 | 443 | 568 |
| PEG conc 2 | 9.6 | 453 | 6273 |
| PEG/Benzonase conc 2 | 9.7 | 462 | 584 |

TABLE 4

| | TCID50 | Protein | DNA |
|---|---|---|---|
| | recovery | recovery | recovery |
| Recovery | [%] | [%] | [%] |
| Harvest | 100 | 100.0 | 100.0 |
| Harvest Clarified | 65 | 23.2 | 50.0 |
| Harvest | 105 | 22.5 | 9.5 |
| clarified/Benzonase | | | |
| PEG conc 1 | 54 | 18.5 | 16.7 |
| PEG/Benzonase conc 1 | 43 | 18.2 | 1.6 |
| PEG conc 2 | 44 | 18.7 | 17.8 |
| PEG/Benzonase conc 2 | 54 | 19.0 | 1.7 |

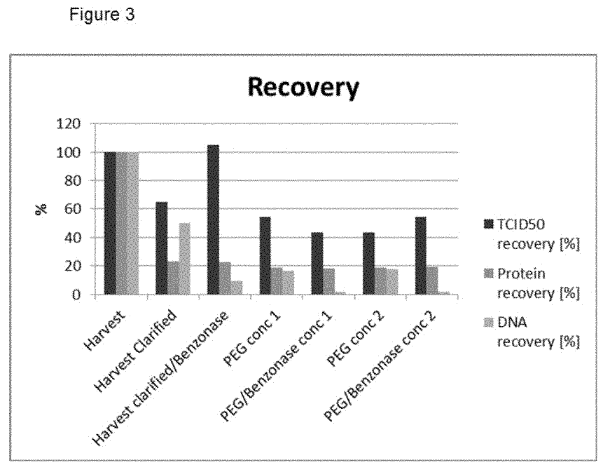

FIG. 3 shows the comparison of recovery of Influenza Type B deINS 106-P2A-GFP using PEG, Benzonase+PEG

Experiment 3

Influenza A deINS106-P2A-E6E7 virus was grown in Vero cells seeded in tissue culture flasks. The virus harvest was clarified by centrifugation for 15 min at 4.100 g and 22° C.

The clarified harvest was treated with Benzonase (20 U/ml for 2 h at RT). Virus precipitation was performed by incubation for 24 h at 4° C. with 8% PEG (w/v) followed by 30 min centrifugation at 4.100 g and 4° C. PEG precipitation was performed in duplicate.

Finally, the precipitated virus was resuspended in virus reconstitution buffer (VRB) at 1/10th of the initial harvest volume resulting in a 10-fold concentrate.

Analyses

Infectious titers were determined by TCID50 assay in Vero cells. Total protein quantification was done in triplicates using a Biorad Bradford assay. For each matrix (OptiPro and VRB) an 8-point standard curve was run in parallel. DNA quantification was done in triplicates using a Picogreen assay. For each matrix (OptiPro and VRB) an 8-point standard curve was run in parallel.

TABLE 5

| | TCID50 | Protein | DNA |
|---|---|---|---|
| Titer/concentration | [log/ml] | [μg/ml] | [ng/ml] |
| Harvest | 8 | 52 | 5414 |
| Harvest Clarified | 8.1 | <2.5 | 1908 |
| Harvest | 8.1 | <2.5 | <122 |
| clarified/Benzonase | | | |
| PEG conc | 8.9 | 25 | 214 |

TABLE 6

| | TCID50 | Protein | DNA |
|---|---|---|---|
| | recovery | recovery | recovery |
| Recovery | [%] | [%] | [%] |
| Harvest | 100 | 100.0 | 100.0 |
| Harvest Clarified | 141 | <4.8 | 35.2 |
| Harvest | 152 | <4.8 | <2.3 |
| clarified/Benzonase | | | |
| PEG conc | 98 | 4.8 | 0.4 |

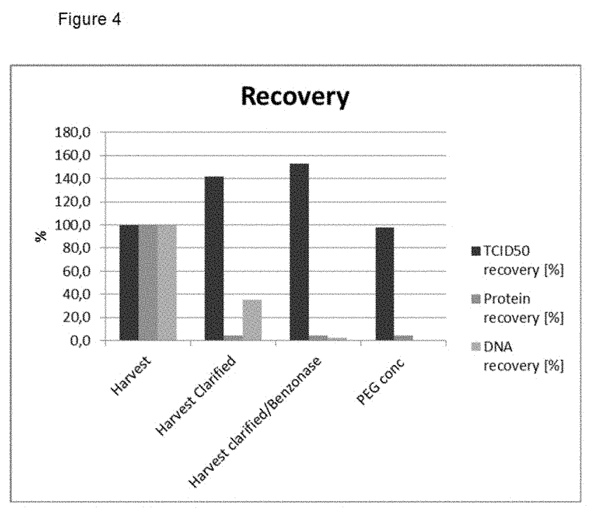

FIG. 4 shows the recovery of Influenza A deINS106-P2A-E6E7 using Benzonase+PEG

1. A method for purifying virus particles from host cells infected with virus, comprising the following steps in the indicated order:
a) cultivating said host cells in a cell culture medium under conditions in which the virus particles are released into the cell culture medium,
b) separating supernatant comprising the virus particles from cell debris and cells by centrifugation,
c) incubating said supernatant with a nuclease,
d) precipitating the virus particles with polyethylene glycol (PEG),
e) centrifuging the virus particles, and
f) resuspending precipitated virus particles.

2. The method according to claim 1, wherein the nuclease is an endonuclease.

3. The method according to claim 1, wherein the virus is an RNA virus.

4. The method according to claim 1, wherein the host cells are provided without previous lysis treatment.

5. The method according to claim 1, wherein centrifugation is at least 2,000 g, at least 3,000 g, at least 4000 g, or at least 4100 g.

6. The method according to claim 1, wherein at least 7.5%, PEG is added.

7. The method according to claim 1, wherein the PEG has an average molecular weight of from about 5,000 (PEG5000) grams per mole to about 15,000 (PEG15000) grams per mole.

8. The method according to claim 1, wherein the virus is precipitated with PEG in the presence of sodium chloride.

9. The method according to claim 1, wherein the virus-infected host cells and the virus are not freeze thawed.

10. The method according to claim 1, wherein at least 50%, at least 60%, at least 70%, at least 80%, at least 85%, at least 90%, at least 95%, or at least 99% host cell protein is removed.

11. The method according to claim 1 any one of claims 1 to 10, wherein at least 50%, at least 60%, at least 70%, at least 80%, at least 85%, at least 90%, at least 95%, or at least 99% of the host cell DNA is removed.

12. The method according to claim 1, wherein the resuspended virus is sterile filtered.

13. The method according to claim 1, wherein the host cells are mammalian cells.

14. The method according to claim 2, wherein the nuclease is an endonuclease from Serratia marcescens.

15. The method according to claim 3, wherein the virus is an influenza virus.

16. The method according to claim 15, wherein the virus is an influenza A, B or C virus.

17. The method according to claim 6, wherein at least 8% PEG is added.

18. The method according to claim 7, wherein the PEG has an average molecular weight of about 6,000 (PEG 6000) grams per mole.

19. The method according to claim 13, wherein the host cells are Vero cells.
